(19) United States
(12) Patent Application Publication
(10) Pub. No.: US 2009/0254192 A1
(43) Pub. Date: Oct. 8, 2009

(54) BONE FILLER AND METHOD FOR FABRICATING THE SAME

(75) Inventors: Kwang Bum Park, Daegu (KR); Kyoung Ho Ryoo, Gwangju (KR); Seok Kyu Choi, Busan (KR); Dong Jun Yang, Daegu (KR); Hyun Wook An, Gyeongsangbuk-do (KR); Jae Won Kim, Gyeongsangbuk-do (KR)

Correspondence Address: CANTOR COLBURN, LLP, 20 Church Street, 22nd Floor, Hartford, CT, 06103, US

(73) Assignee: MEGAGEN IMPLANT CO., LTD., Gyeongsan-si (KR)

(21) Appl. No.: 12/440,659

(22) PCT Filed: Aug. 3, 2007
(86) PCT No.: PCT/KR2007/003749

§ 371 Date: Mar. 10, 2009

(30) Foreign Application Priority Data
Sep. 11, 2006 (KR) 10-2006-0087700

(51) Int. Cl. A61F 2/28; C04B 35/64

(52) U.S. Cl. 623/23.5; 264/610

(57) ABSTRACT

Provided are a bone filler for orthopedic and dental applications and a method of fabricating the same. The bone filler includes a pillar-like body having at least one through hole. In addition, the method of fabricating a bone filler includes preparing a slurry including a bioactive material, pressing the slurry to form a pillar-like body including at least one through hole, and calcining the body.

### TECHNICAL FIELD

The present invention relates to a bone filler and a method of fabricating the same, and more particularly, to a bone filler used for orthopedic and dental applications and a method of fabricating the same.

### BACKGROUND ART

Porous bioactive ceramics are attractive for bone fillers used for orthopedic and dental applications. Dense bioactive ceramics are attached to bones, but cannot be used as a substitute for bones since they include insufficient void space for bone growth and remain in living organism, and thus restrict bone growth. However, when the bioactive ceramics are porous, bones can grow into the pores and thus the bioactive ceramics can be completely combined with bones.

Generally, net-like bone fillers in which pores are three-dimensionally connected with each other have been used. However, in the bone filler having three-dimensionally connected pores, the pore size cannot be precisely defined, the pores may be blocked when the porosity is too low, and strength of the bone filler may be decreased since porosity increase is limited.

### DISCLOSURE

### Technical Problem

The present invention provides a bone filler having excellent biocompatibility, stable mechanical strength and high bone forming efficiency.

The present invention also provides a method of fabricating a bone filler having excellent biocompatibility, stable mechanical strength and high bone forming efficiency.

### Advantageous Effects

A bone filler according to the present invention has excellent biocompatibility, stable mechanical strength and high bone forming efficiency by including a porous body having a through hole.

In addition, damages of a bone defect region can be inhibited and adverse effects caused by fragments separated from the bone filler can be decreased by polishing the surface of the bone filler.

In addition, according to a method of fabricating a bone filler according to the present invention, the bone filler can be calcined at a lower temperature by pressing a slurry including a bioactive material to form the bone filler in a constant shape. Thus, possibility of transformation of materials forming the bone filler can be decreased, and the manufacturing costs can be reduced by decreasing the amount of bone filler loss unnecessarily incurred.

### DESCRIPTION OF DRAWINGS

FIGS. 1 to 3 show perspective views of bone fillers according to an embodiment and modified embodiments of the present invention;

FIG. 4 shows a perspective view of a bone filler according to another embodiment of the present invention;

FIG. 5 shows a flow chart of a method of fabricating a bone filler according to an embodiment of the present invention;

FIG. 6 shows a cross-sectional view of a bone filler forming apparatus used in a method of fabricating a bone filler according to an embodiment of the present invention;

FIG. 7 shows a cross-sectional view of the bone filler forming apparatus of FIG. 6 in which a piston is raised to the highest position;

FIG. 8 shows a cross-sectional view of the bone filler forming apparatus of FIG. 6 when a slurry is injected;

FIG. 9 shows a side view of the bone filler forming apparatus of FIG. 8;

FIG. 10 shows a cross-sectional view of a part of the bone filler forming apparatus of FIG. 6 when the bone filler is formed;

FIG. 11 shows a flow chart of a method of fabricating a bone filler according to another embodiment of the present invention;

FIG. 12 shows a cross-sectional view of a polishing device used in a method of fabricating a bone filler according to another embodiment of the present invention;

FIG. 13 shows a scanning electron microscope (SEM) image of a bone filler polished by the polishing device of FIG. 12;

FIG. 14 is a graph illustrating the results from cell toxicity test of bone fillers; and

FIG. 15 is a graph illustrating the results from cell growth test of bone fillers.

### BEST MODE

According to an aspect of the present invention, there is provided a bone filler comprising a pillar-like body including at least one through hole.

Here, a cross-section of the through hole may be a circle or a polygon.

The body may have a cylindrical or polygonal pillar shape, and include micropores.

The surface of the body may be polished.

The body may include at least one bioactive material selected from the group consisting of hydroxyapatite, tricalcium phosphate, monocalcium phosphate, tetracalcium hexaphosphate, calcium sulfate, calcium carbonate, bioactive glass and glass ceramics.

In addition, the body may have a diameter in the range of 100 to 1000 μm, and the through hole may have a diameter less than 60% of the diameter of the body.

Such a bone filler may have a porosity of about from 50 to 90%.

According to another aspect of the present invention, there is provided a method of fabricating a bone filler, the method comprising: preparing a slurry including a bioactive material; pressing the slurry to form a pillar-like body including at least one through hole; and calcining the body.

The bioactive material may be at least one selected from the group consisting of hydroxyapatite, tricalcium phosphate, monocalcium phosphate, tetracalcium hexaphosphate, calcium sulfate, calcium carbonate, bioactive glass and glass ceramics.

The slurry may further comprise a pore generator which is at least one selected from the group consisting of polymethylmethacrylate, starch and naphthalene.

The pressing step may further comprise drying the pillar-like body in which the pillar-like body may be dried at a temperature in the range of 100 to 200° C.

The pressing step or the calcining step may further comprise cutting the pillar-like body to a desired length.

A cross-section of the through hole may be a circle or a polygon.

The body may have a cylindrical or polygonal pillar shape.

The body may have a diameter in the range of 100 to 1000 μm, and the through hole may have a diameter less than 60% of the diameter of the body.

The bone filler may have a porosity of about from 50 to 90%.

The body may be calcined at a temperature in the range of 1000 to 1300° C. during the calcining step.

The method may further comprise polishing the body after the calcining step, and the polishing step may be performed in water or an organic solvent having a density which is substantially identical to or less than a density of water. The organic solvent may be at least one solvent selected from the group consisting of ethanol, methanol and acetone, and preferably methanol.

### MODE FOR INVENTION

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art without departing from the spirit and scope of the present invention as defined by the claims. The same reference numerals indicate the same elements.

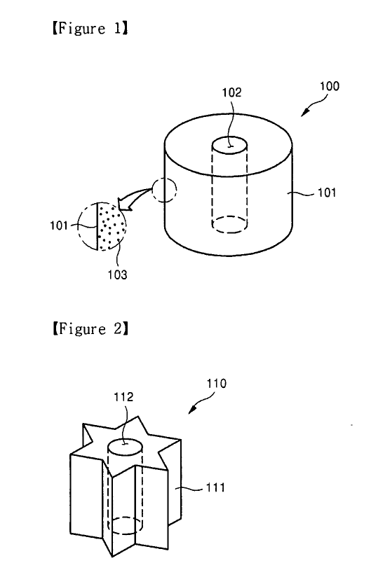

Hereinafter, a bone filler according to the present invention will be described in detail with reference to FIGS. 1 to 3. FIGS. 1 to 3 show perspective views of bone fillers according to an embodiment and modified embodiments of the present invention.

As shown in FIGS. 1 to 3, bone fillers 100, 110 and 120 according to an embodiment and modified embodiments of the present invention include pillar-like bodies 101, 111 and 121 including at least one through holes 102, 112 and 122.

The bodies 101, 111 and 121 of the bone fillers 100, 110 and 120 which directly contact with cells in a bone defect region when the bone fillers 100, and 120 are transplanted into a living body can induce growth of new bones in the bone defect region. A part of or the entire bodies 101, 111 and of the bone fillers 100, 110 and 120 are gradually degraded, and newly induced bones are filled in the space formed by the degradation and replace the bodies 101, 111 and 121.

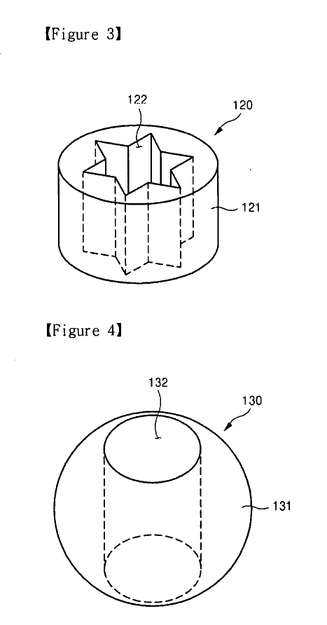

The bodies 101, 111 and 121 of the bone fillers 100, 110 and 120 may have a cylindrical or polygonal pillar shape. That is, the body of the bone fillers 100, 110 and 120 may be a cylinder as shown in FIGS. 1 and 3, a pillar having an oval-shaped cross-section (not shown) or a pillar having a polygonal-shaped cross-section such as a triangle, a square, a pentagon and a star 111 as shown in FIG. 2

In addition, since micropores are formed in the bodies 101, 111 and 121 of the bone fillers 100, 110 and 120, the bodies 101, 111 and 121 of the bone fillers 100, 110 and 120 have porous structures. When the bone fillers 100, 110 and 120 are transplanted into the bone defect region, blood passes through the micropores 103 formed in the bodies 101, 111 and 121 of the bone fillers 100, 110 and 120 to effectively help bone formation.

The bone fillers 100, 110 and 120 include at least one through holes 102, 112 and 122 in the bodies 101, 111 and 121. The through holes 102, 112 and 122 are macropores having a relatively large size compared to the micropores 103 and formed in the bodies 101, 111 and 121. When the bone fillers 100, 110 and 120 are transplanted into the bone defect region, body fluids including blood pass through the through holes 102, 112 and 122 like the micropores 103 formed in the bodies 101, 111 and 121 of the bone fillers 100, 110 and 120 to effectively help bone formation, and the through holes 102, 112 and 122 provide sites on which newly formed bone cells are adsorbed to prevent the newly formed bone cells from being separated.

The through holes 102, 112 and 122 have a pillar shape penetrating the bone fillers 100, 110 and 120. The cross-section of the through holes 102, and 122 may be a circle or a polygon. That is, the through holes 102, and 122 of the bone fillers 100, 110 and 120 may be a cylinder as shown in FIG. 1 or a pillar having an oval-shaped cross-section (not shown). In addition, the through holes 102, 112 and 122 of the bone fillers 100, 110 and may be a pillar having a polygonal-shaped cross-section such as a triangle, a square, a pentagon and a star 122 as shown in FIG. 3. Here, the shape of an outside circumference of the bodies 101, 111 and 121 of the bone fillers 100, 110 and 120 may be the same as or different from that of the through holes 102, 112 and 122.

In addition, the number of the through holes 102, 112 and 122 of the bone fillers 100, 110 and 120 are not limited, but can be controlled according to porosity, strength and size of desired bone fillers 100, 110 and since too many through holes 102, 112 and 122 may weaken the strength of the bone fillers 100, 110 and 120.

The bodies 101, 111 and 121 of the bone fillers 100, 110 and 120 may include a material having excellent bioactivity, and include at least one bioactive material selected from the group consisting of hydroxyapatite, tricalcium phosphate, monocalcium phosphate, tetracalcium hexaphosphate, calcium sulfate, calcium carbonate, bioactive glass and glass ceramics.

The bone fillers 100, 110 and 120 may have a length in the range of about 0.5 to 2 mm and a diameter in the range of about 100 to 1000 μm, but the length and diameter are not limited. The bone fillers 100, 110 and 120 have a relatively small size in order not to be restricted by the size of the bone defect region to which the bone fillers 100, 110 and 120 are applied. That is, the small-sized bone fillers 100, 110 and 120 can be efficiently transplanted into a narrow bone defect region. Here, the through holes 102, and 122 of the bone fillers 100, 110 and 120 may have a diameter less than 60%, and preferably less than about 50%, of the diameter of the bodies 101, 111 and 121 of the bone fillers 100, 110 and 120.

The bone fillers 100, 110 and 120 having the micropores 103 and the through holes 102, 112 and 122 can have a porosity of about from 50 to 90%. A bone filler according to another embodiment of the present invention will now be described with reference to FIG. 4. FIG. 4 shows a perspective view of a bone filler according to another embodiment of the present invention.

The bone filler shown in FIG. 4 is the same as those described above except that the surface of the bone filler is polished. Thus, the bone filler of FIG. 4 will be described based on differences.

The bone filler 130 of FIG. 4 includes a pillar-like body 131 including at least one through hole 132. The surface of the bone filler 130 is polished and thus the bone filler 130 has a relatively smooth surface. Sharp edges of the bone filler 130 are also polished and thus the edges also have a smooth surface.

The bone filler 130 having the polished surface is transplanted into the bone defect region and help to form new bones without damaging cells of the bone defect region. The bone filler 130 is not easily broken since the edges are removed by the polishing process.

FIG. 4 only describes a bone filler 130 including a cylindrical body 131 having a cylindrical through hole 132. However, the surface of the bone filler including a cylindrical body having a through hole with a polygonal-shaped cross-section, a body with a polygonal-shaped cross-section having a cylindrical through hole, or a body with a polygonal-shaped cross-section having a through hole with a polygonal-shaped cross-section, although they are not shown, can also be polished, and thus the bone filler may have a smooth surface without sharp edges.

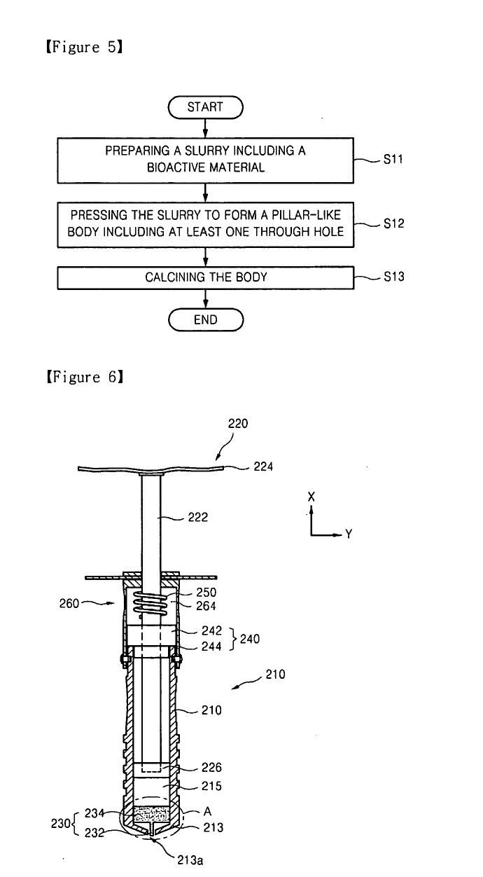

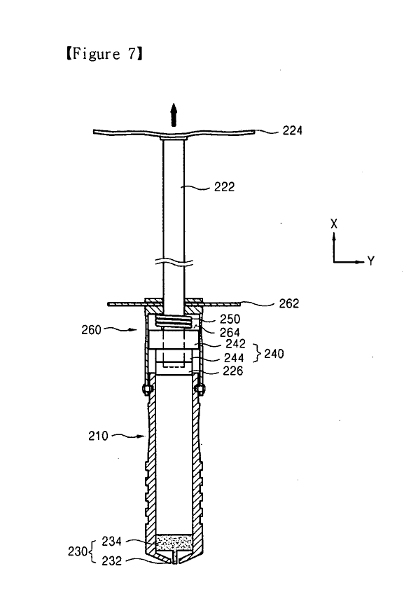

A method of fabricating a bone filler according to an embodiment of the present invention will be described. FIG. 5 shows a flow chart of a method of fabricating a bone filler according to an embodiment of the present invention.

As shown in FIG. 5, a slurry including a bioactive material is prepared S11.

The bioactive material which is a material having activity in a living organism and inducing new-bone formation may be at least one selected from the group consisting of hydroxyapatite, tricalcium phosphate, monocalcium phosphate, tetracalcium hexaphosphate, calcium sulfate, calcium carbonate, bioactive glass and glass ceramics.

The bioactive material may include a pore generator which will be removed by calcining the body to form micropores. The bone filler can have a porous structure by the formed micropores, and the porous bone filler can efficiently induce new-bone formation since blood can easily enter the micropores when transplanted into the bone defect region.

The pore generator may be at least one selected from the group consisting of polymethylmethacrylate (PMMA), starch and naphthalene.

The bioactive material may further include a slurry-forming solvent, a calcining additive that improves calcination capability, a foaming agent that helps generating pores, and the like. The slurry-forming solvent may be cast-oil, the calcining additive may be poly ethylene glycol (PEG), and the foaming agent may be PMMA.

A mixing device, for example a tank-type mixing device in which a mixture is stirred using an impeller operated by rotating force from a motor for a uniform composition of the mixture, may be used in order to form a slurry of the bioactive material. However, the mixing device used in the method of fabricating a bone filler according to an embodiment of the present invention is not limited thereto, and any mixing device that can achieve the objects of the present invention will be used.

Then, the slurry is pressed to form a pillar-like body including at least one through hole S12 of FIG. 5.

In order to press the slurry to form the pillar-like body including at least one through hole, a bone filler forming apparatus may be used. FIG. 6 shows a cross-sectional view of a bone filler forming apparatus used in a method of fabricating a bone filler according to an embodiment of the present invention.

As shown in FIG. 6, the bone filler forming apparatus 200 which is a device pressing the slurry to form a desired shape includes a cylinder 210 in which the slurry is filled, a piston 220 pressing the slurry to discharge the slurry, a through hole-forming unit 230 that forms a through hole in the discharged slurry, a pipe 240 combined with the upper part of the cylinder 210 to support the piston 220, an elastic body 250 providing elasticity to the pipe 240, and a pipe receiving unit 260 receiving the pipe 240 and the elastic body 250.

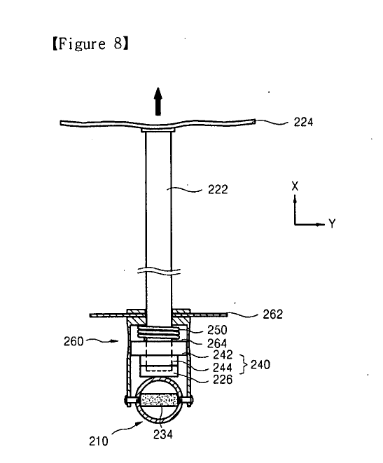

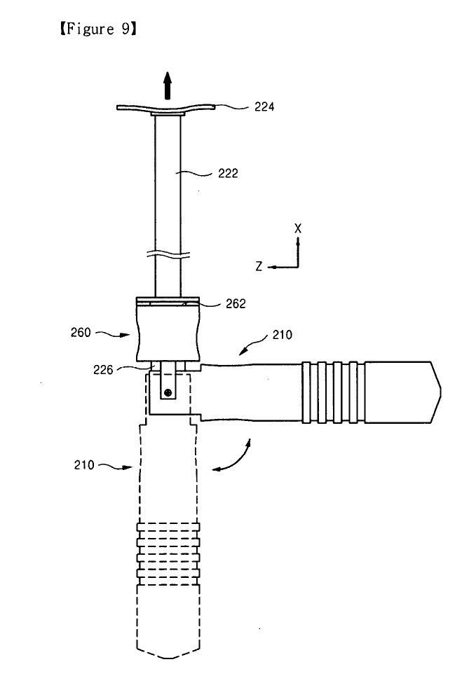

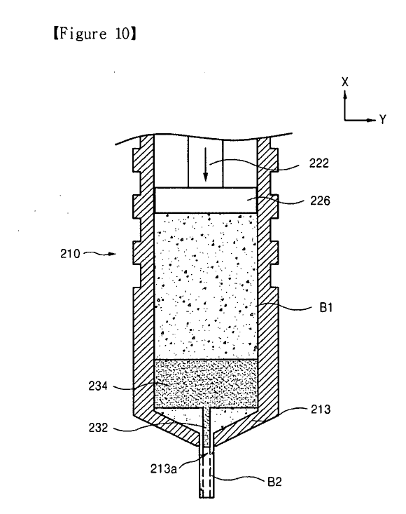

A method of pressing the slurry using the bone filler forming apparatus 200 will be described with reference to FIGS. 7 to 10. FIG. 7 shows a cross-sectional view of the bone filler forming apparatus in which a piston is raised to the highest position. FIG. 8 shows a cross-sectional view of the bone filler forming apparatus when the slurry is injected. FIG. 9 shows a side view of the bone filler forming apparatus of FIG. 8. FIG. 10 shows a cross-sectional view of a part of the bone filler forming apparatus when the bone filler is formed.

As shown in FIG. 7, when a piston rod 222 is sufficiently raised in +X direction using a piston grip 224 with fingers, a packing 226 which is combined with the lower part of the piston rod 222 contacts with a lower pipe 244. When the piston rod 222 is further raised in +X direction, the lower pipe 244 combined with the cylinder 210 is separated from the cylinder 210. Here, the elastic body 250 is pressed to provide elasticity to the upper pipe 242 in −X direction.

When the cylinder 210 is rotated by 90 degrees around the Y axis, the length direction of the piston rod 222 intersects the length direction of the cylinder 210 at a right angle as shown in FIGS. 8 and 9. Accordingly, even if the external force that raises the piston rod 222 in +X direction is removed, the piston 220 and the cylinder 210 can be maintained in a right angle configuration since the pipe 240 is forced in −X direction by the pressed elastic body 250.

When the piston 220 and the cylinder 210 are maintained in a right angle configuration due to the elasticity of the elastic body 250, the slurry may be injected into a slurry storing room 215 in the cylinder 210.

When the slurry is injected, the piston rod 222 is raised in +X direction again, the cylinder 210 is rotated by 90 degrees around the Y axis to locate the piston rod 222 and the cylinder 210 in the same direction. Then, the piston rod 222 is moved downward in −X direction to combine the lower pipe 244 with the cylinder 210 and the lower part of the piston rod 222 is disposed in the cylinder 210. Thus, the bone filler forming apparatus 200 returns the same state shown in FIG. 7.

Then, as shown in FIG. 10, when the slurry B₁ filled in the cylinder 210 is pressed downward by the piston rod 222, the slurry B₁ is transferred downward by the pressure from the cylinder 210 and the slurry B₂ is discharged through a discharge pipe 213a to be formed into a pillar-like body including a through hole. The external diameter of the formed body is the same as that of a discharge nozzle 213, and the internal diameter of the body is the same as that of a through hole core 232. The shape and size of the through hole may be adjusted to a desired level by regulating the shape and size of the through hole core 232.

Then, the formed pillar-like body including the through hole is dried to have strength suitable for cutting.

The drying may be performed by disposing the body in a heating means such as a hot air drier or a vacuum drier at a temperature in the range of about 100 to 200° C. for about 0.5 to 1 hour.

Then, the dried body is cut to a desired size. For examples, the dried slurry may be cut into pieces to have a length in the range of bout 0.5 to 2 mm. The body is cut in a relatively small size in order not to be affected by the size of the bone defect region, that is, in order to efficiently be transplanted into a narrow bone defect region. The cutting may be performed after the drying step or after a calcining step which will be described later.

The cutting device may be a precise cutting device such as a laser cutting device and a water jet cutting device. However, any cutting device that can achieve the objects of the present invention can also be used.

Then, the pillar-like body including a through hole is calcined S13 of FIG. 5.

The pillar-like body including a through hole is calcined at a high temperature to provide the bone filler with a constant hardness and to burn and remove the pore generator for three-dimensional formation of micropores. Here, the calcination may be performed at a temperature in the range of about 1000 to 1300° C. for about 1 to 2 hours.

Since the pillar-like body is pressed by the bone filler forming apparatus, it can be calcined at a temperature about 100 to 200° C. lower than calcination of a non-pressed body. The calcination is a process of providing particles constitutioning the body with binding energy. Since the body pressed before calcination has a dense structure, a desired hardness of the body can be obtained by applying relatively low energy.

When the body is calcined at a relatively low temperature, the possibility of transformation of the bioactive material included in the body is remarkably decreased. When a part of the bioactive material in the body is transformed, degradation rates between the transformed bioactive material and the non-transformed bioactive material may be different. Thus, when the bone filler including the bioactive material, a part of which is transformed, is transplanted into the bone defect region, the bone generating efficiency may be decreased since the bone filler cannot be degraded in an appropriate rate. Thus, the bone generating efficiency can be increased by minimizing transformation of the bioactive material by decreasing the calcining temperature.

As described above, a bone filler according to an embodiment of the present invention includes a porous structure in the body having at least one through hole. Thus, the bone filler functions as a path of blood and bones to facilitate formation of new bones and provides sites for the adsorption of the new bones to prevent the new bones from being separated when transplanted into the bone defect region.

In addition, the bone filler can be calcined at a relatively lower temperature since it is formed by pressing. Thus, the bone filler can have a constant degradation rate in a living body since it has a low possibility of transformation.

In addition, the size of the bone filler, the size and number of the through hole in the bone filler can be precisely controlled. A conventional bone filler selecting step in which the bone filler is pulverized and bone fillers having a desired size to be transplanted into the bone defect region are selected is not necessary since the bone filler has a constant size. Thus, the amount of bone filler loss that is incurred by the conventional method requiring the bone filler selecting step can be decreased.

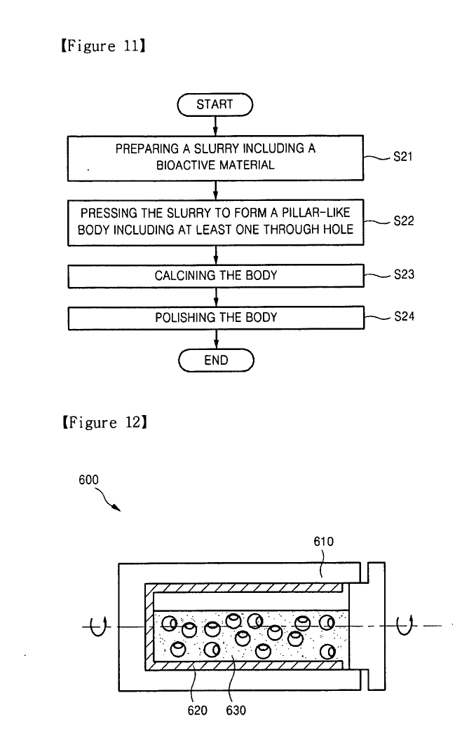

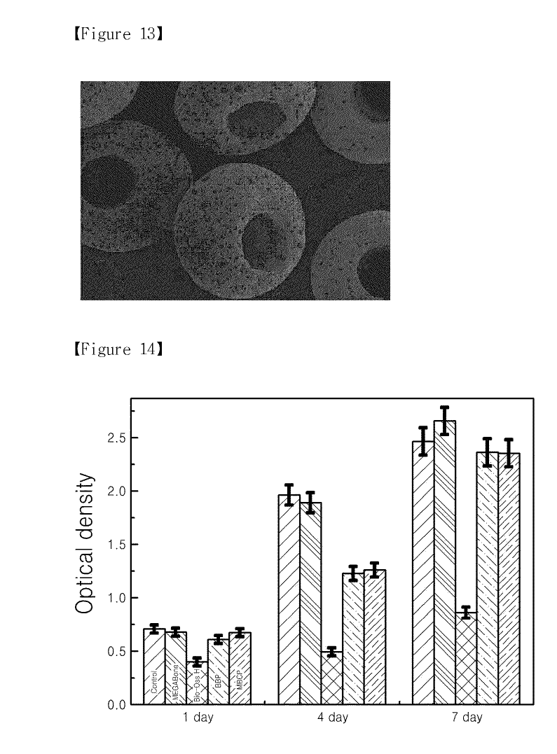

A method of fabricating a bone filler according to another embodiment of the present invention will be described with reference to FIGS. 11 to 13. FIG. 11 shows a flow chart of a method of fabricating a bone filler according to another embodiment of the present invention. FIG. 12 shows a cross-sectional view of a polishing device used in a method of fabricating a bone filler according to another embodiment of the present invention. FIG. 13 shows a scanning electron microscope (SEM) image of a bone filler polished by the polishing device.

The method of fabricating a bone filler according to another embodiment of the present invention is substantially identical to the method described above except that the method further includes a polishing process. Thus, the method of fabricating a bone filler will be described based on the difference.

First, as shown in FIG. 11, a slurry including a bioactive material is prepared S21, the slurry is pressed to form a pillar-like body including at least one through hole S22, and the pillar-like body is calcined S23.

Then, the calcined body is polished S24. During the polishing step, the surface and edges of the body are polished to form smooth surface and rounded edges. When the polished bone filler is transplanted into the bone defect region, cell damages caused by sharp portions of the bone filler can be prevented during the new-bone formation and fixation since sharp portions are removed. In addition, adverse effects caused by fragments separated from the bone filler can be prevented since fragile edges are already removed.

The polishing may be performed using a polishing device 600 including a container 610 in which the bone filler is filled, a cover 620 sealing the container 610 and a polishing plate 630 polishing edges of the bone filler by friction as shown in FIG. 12.

In order to polish the bone filler, the bone filler is filled in the container 610 of the polishing device 600, and the surface and edges of the bone filler are polished by contacting the bone filler with the polishing plate 630 formed of a metal such as stainless steel on which diamond is uniformly distributed by rotating the container 610.

Here, the polishing step may be performed in water or an organic solvent 640. That is, water or the organic solvent 640 may be filled in the container 610, and the polishing may be performed while the bone filler is floating on water or the organic solvent 640. Water or the organic solvent 640 functions as a cushion and protect the bone filler from impact by collisions, and also soften the surface of the bone filler.

The density of the organic solvent may be substantially identical to or lower than that of water in order to polish the bone filler which is three-dimensionally distributed in the organic solvent. The organic solvent may be ethanol, methanol and acetone, and preferably methanol.

As shown in FIG. 13, the polished bone filler can have excellent properties by having smooth surface, decreasing the risk of cell damages when transplanted into the bone defect region since the edges are removed, and decreasing fractions of the bone.

The present invention will be described in more detail with reference to the examples below, but is not limited thereto. The following examples are for illustrative purposes only and are not intended to limit the scope of the invention.

### Experimental Example 1

### Preparation of Bone Filler

A 0.2 M aqueous calcium nitrate trihydrate [Ca(NO₃)₂.H₂O] solution and a 0.2 M aqueous triethyl phosphite [P(OC₂H₅)₃] solution were prepared. An aqueous calcium nitrate solution and an aqueous ammonium phosphate solution were prepared to adjust the molar ratio of Ca/P to 1.0 to 2.0. The aqueous solutions were respectively treated with 1 l methyl alcohol (98%, Duksan Chemicals Co.) and 1 l of hydrochloric acid (35%, Duksan Chemicals Co.) to facilitate the reaction, and the calcium nitrate solution was mixed with the ammonium phosphate solution.

The mixture was stirred at room temperature for 1 hour, gradually dried to prepare powder. The powder was pulverized using a mortar and dried in a drier (WOF-155, Daihan Scientific Co., Ltd.) at 150° C. for about 12 hours, and then calcined at 800° C. for 1 hour to prepare a calcium phosphate compound. 500 ml of castor oil (Duksan Chemicals Co.), 400 of polyethylene glycol (1 kg, Duksan Chemicals Co.), and 500 g of polyvinyl alcohol (Duksan Chemicals Co.) were added to the powder and mixed to prepared a slurry.

The mixture in a slurry state was injected into a bone filler forming apparatus, and a long pillar-like body including a through hole was prepared by pressing a piston.

Then, the pillar-like body was dried in a drier at 100° C. for 1 hour, and placed in an alumina pot. The alumina pot was maintained in an electric furnace (UP-350, Thermonik Inc.) at 1200° C. for 2 hours to obtain a calcined body. The temperature was increased and decreased by 5° C./min.

The calcined body was cut into pieces having a desired size in the range of 1.0 to 2.0 mm by a cutter.

1 l of methanol (Duksan Chemicals Co.) which is 60% by volume of the cut bone filler was filled in the polishing container and polishing was performed for 24 hours. The polished bone filler was washed with methanol, and then washed with an ultrasonic cleaner (Power Sonic 420, Hwashin Instrument Co., Ltd.) for 1 hour. The washed bone filler was sterilized in a dryer at 300° C. for 1 hour, and stored in a sealed container.

### Experimental Example 2

### Cell Toxicity Test of Bone Filler

MC3T3E1 cells which are osteoblasts of albino rats were cultured using a Dulbecco's modified eagles media (DMEM) including 10% fetal bovine serum in order to test cell toxicity of a bone filler.

Cells cultured in a mono-layer in a cell culture flask were treated with trypsin-EDTA to collect the cells. The number of cells was calculated using a hemocytometer, and 2×104 cells were cultured in 24-well culture flasks.

Cells that does not include a bone filler was used as a control group. Cells including a bone filler MEGABone prepared in Experimental Example 1 and cells including three types of bone fillers (Bio-OSS (Geistlich biomaterials, Swiss), BBP (Oscotec, Inc, Republic of Korea), and MBCP (Biomatlant, French)) were used as experimental groups. 25 mg of each of the cells was cultured in a culture flask using 5% CO₂ incubator for 1, 4 and 7 days.

After culturing the cells, the culture medium was removed and the cells were washed twice with 1 mg of phosphate-buffered saline (pH7.0). 1 ml of a culture medium not including fetal bovine serum was added thereto, and 200 μl of MTT (3-(4,5-dimethylithiazol-2-yl)-2,5-diphenly tetrazolium bromide) reagent (pH7.0, 5 mg/ml in PBS) was added thereto. Then, the cells were cultured in a 5% CO₂ incubator at 37° C. for 3 hours without light.

Then, the supernatant was removed, and 750 μl of dimethyl sulfoxide (DMSO) was added to each of the culture flasks. 150 μl of each of the mixtures was placed in a 96-well culture flask to measure absorbance at 570 nm using a ELISA reader (Bio-rad). Cell toxicity was obtained by subtracting absorbance measured at a reference wavelength 620 nm from the absorbance at 570 nm, and the results are shown in FIG. 14.

As shown in FIG. 14, upon comparing optical density of the control group not including the bone filler with optical density of the MEGABone experimental group including the bone filler according to an embodiment of the present invention, optical density of the MEGABone experimental group was similar to or higher than that of the control group on the fourth day and seventh day of the experiment. On the contrary, optical density of the Bio-OSS experimental group was far lower than that of the control group. Optical densities of the BBP experimental group and the MBCP experimental group were lower than those of the control group on the fourth day of the experiment, and similar to those of the control group from the seventh day of the experiment.

Based on the cell toxicity test of the MEGABone experimental group including the bone filler according to the present invention with reference to the results of FIG. 14, the bone filler according to the present invention does not inhibit cell growth but facilitate cell growth.

### Experimental Example 3

### Cell Growth Test of Bone Filler

MC3T3E1 cells which are osteoblasts of albino rats were cultured using a Dulbecco's modified eagles media (DMEM) including 10% fetal bovine serum. Cells cultured in a mono-layer in a cell culture flask were treated with trypsin-EDTA to collect the cells. The number of cells was calculated using a hemocytometer, and 2×104 cells were cultured in 24-well culture flasks.

Cells that does not include a bone filler was used as a control group. Cells including a bone filler MEGABone prepared in Experimental Example 1 and cells including three types of bone fillers (Bio-OSS (Geistlich biomaterials, Swiss), BBP (Oscotec, Inc, Republic of Korea), and MBCP (Biomatlant, French)) were used as experimental groups. 25 mg of each of the cells was cultured in a culture flask at 37° C. using 5% CO₂ incubator for 3, 5 and 7 days.

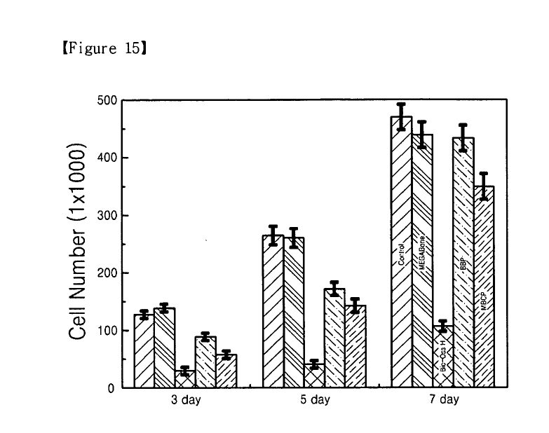

After culturing the cells, the culture medium was removed and the cells were washed twice with 1 ml of phosphate-buffered saline (pH7.0). The cells were treated with trypsin-EDTA to collect the cells, and the numbers of cells of the control group and the experimental groups (MEGABone, Bio-OSS, BBP and MBCP) were calculated using a hemocytometer. The results are shown in FIG. 15.

As shown in FIG. 15, upon comparing the cell number of the control group not including the bone filler with the cell number of the MEGABone experimental group including the bone filler according to an embodiment of the present invention, the cell number of the MEGABone experimental group had an increase tendency similar to the cell number of the control group on the fourth day and seventh day of the experiment. On the contrary, the Bio-OSS experimental group had a far lower increase in the cell number compared with the control group. The BBP and MBCP experimental groups had a lower increase in the cell number than the control group on the fourth day of the experiment, but a similar increase to the control group from the seventh day of the experiment.

According to the results of FIG. 15, cells can relatively quickly grow when the MEGABone including the bone filler of the present invention is applied.

While the present invention has been particularly shown and described with reference to exemplary embodiments thereof, it will be understood by those of ordinary skill in the art that various changes in form and details may be made therein without departing from the spirit and scope of the present invention as defined by the following claims.

### INDUSTRIAL APPLICABILITY

A bone filler according to the present invention has excellent biocompatibility, stable mechanical strength and high bone forming efficiency by including a porous body having a through hole.

In addition, damages of a bone defect region can be inhibited and adverse effects caused by fragments separated from the bone filler can be decreased by polishing the surface of the bone filler.

In addition, according to a method of fabricating a bone filler according to the present invention, the bone filler can be calcined at a lower temperature by pressing a slurry including a bioactive material to form the bone filler in a constant shape. Thus, possibility of transformation of materials forming the bone filler can be decreased, and the manufacturing costs can be reduced by decreasing the amount of bone filler loss unnecessarily incurred.

1. A bone filler comprising a pillar-like body including at least one through hole.

2. The bone filler of claim 1, wherein a cross-section of the through hole is a circle or a polygon.

3. The bone filler of claim 1, wherein the body has a cylindrical or polygonal pillar shape.

4. The bone filler of claim 1, wherein the body comprises micropores.

5. The bone filler of claim 1, wherein the surface of the body is polished.

6. The bone filler of claim 1, wherein the body comprises at least one bioactive material selected from the group consisting of hydroxyapatite, tricalcium phosphate, monocalcium phosphate, tetracalcium hexaphosphate, calcium sulfate, calcium carbonate, bioactive glass and glass ceramics.

7. The bone filler of claim 1, wherein the body has a diameter in the range of 100 to 1000 μm.

8. The bone filler of claim 7, wherein the through hole has a diameter less than 60% of the diameter of the body.

9. The bone filler of claim 1, having a porosity of from 50 to 90%.

10. A method of fabricating a bone filler, the method comprising: preparing a slurry including a bioactive material;
pressing the slurry to form a pillar-like body including at least one through hole; and
calcining the body.

11. The method of claim 10, wherein the bioactive material is at least one selected from the group consisting of hydroxyapatite, tricalcium phosphate, monocalcium phosphate, tetracalcium hexaphosphate, calcium sulfate, calcium carbonate, bioactive glass and glass ceramics.

12. The method of claim 10, wherein the slurry further comprises a pore generator.

13. The method of claim 12, wherein the pore generator is at least one selected from the group consisting of polymethylmethacrylate, starch and naphthalene.

14. The method of claim 10, wherein the pressing step further comprises drying the pillar-like body.

15. The method of claim 14, wherein the pillar-like body is dried at a temperature in the range of 100 to 200° C.

16. The method of claim 14, wherein the pressing step or the calcining step further comprises cutting the pillar-like body into a desired length.

17. The method of claim 10, wherein a cross-section of the through hole is a circle or a polygon.

18. The method of claim 10, wherein the body has a cylindrical or polygonal pillar shape.

19. The method of claim 10, wherein the body has a diameter in the range of 100 to 1000 μm.

20. The method of claim 19, wherein the through hole has a diameter less than 60% of the diameter of the body.

21. The method of claim 10, wherein the bone filler has a porosity of from 50 to 90%.

22. The method of claim 10, wherein the body is calcined at a temperature in the range of 1000 to 1300° C. during the calcining step.

23. The method of claim 10, further comprising polishing the body after the calcining step.

24. The method of claim 23, wherein the polishing step is performed in water or an organic solvent having a density which is substantially identical to or less than a density of water.

25. The method of claim 24, wherein the organic solvent is at least one solvent selected from the group consisting of ethanol, methanol and acetone.
